(19) United States
(12) Patent Application Publication
(10) Pub. No.: US 2010/0004516 A1
(43) Pub. Date: Jan. 7, 2010

(54) Method and System for Monitoring Physiological Status

(75) Inventors: You-Wei Teng, Taipei (TW); Yi-Chih Yeh, Taipei (TW); Ming-Feng Li, Taipei (TW)

Correspondence Address: DITTHAVONG MORI & STEINER, P.C., 918 Prince Street, Alexandria, VA, 22314, US

(73) Assignee: Micro-Star Int'l Co., Ltd., Jung-He City (TW)

(21) Appl. No.: 12/358,274

(22) Filed: Jan. 23, 2009

(30) Foreign Application Priority Data
Jul. 1, 2008 (TW) 097124790

(51) Int. Cl. A61B 5/00

(52) U.S. Cl. 600/301

(57) ABSTRACT

A method for monitoring physiological status is applied to a laptop to form a monitoring system. A sensor is disposed on the laptop adjacent to a keyboard of the laptop. The sensor is provided for sensing user's physiological signal and a detection circuit is utilized for converting the physiological signal into a numerical value. Detection software is installed to the laptop and executed for comparing the numerical value with a pre-determined threshold value range and output the numerical value and a comparison result. An alert is issued when the numerical value falls outside the threshold value range.

### CROSS-REFERENCES TO RELATED APPLICATIONS

This non-provisional application claims priority under 35 U.S.C. §119(a) on Patent Application No(s). 97124790 filed in Taiwan, R.O.C. on Jul. 1, 2008, the entire contents of which are hereby incorporated by reference.

### FIELD OF THE INVENTION

The present invention relates to monitoring physiological status of a user, and more particularly to a method and system for monitoring the physiological status of a laptop user.

### BACKGROUND

Currently, going with the development of electronic technology and the growing attention to consumer-directed health care, physiological status monitoring devices are permitted to be used in the domestic environment, and not limited to a professional medical station. For example, instruments such as the sphygmomanometers, electronic clinical temperature sensors, and blood glucose meter kits are now popular in homes. The object of physiological status monitoring is allowing a user to know his physiological condition well, so as to help prevent various potential diseases. For example, a measurement of a user's blood pressure allows him to detect his blood pressure condition earlier to prevent high blood pressure or low blood pressure from causing any potential danger. The basic physiological signal measurements can assist modern medical science to evaluate personal physiological status so as to carry out a fundamental requirement of disease prevention.

The current physiological signal measuring instrument, taking a measurement of heartbeat or electrocardiogram as an example, uses electrodes or conductive pads attached onto a human skin to detect a hypodermic current variation. For example, a “PORTABLE PHYSIOLOGY MONITORING DEVICE AND SYSTEM” disclosed in Taiwan Patent No. M298980, a “BLUETOOTH EARPHONE FOR MONITORING HEARTBEAT AND A RECORDING OR DISPLAYING ELECTRONIC DEVICE” disclosed in Taiwan Patent No. M286024, and a “PAD-FREE ELECTROCARDIOGRAM MEASURING APPARATUS AND METHOD” disclosed in Taiwan Patent No. I234449 all adopt an electrode or conductive pad to detect a physiological signal and transmit it to a specific device. Otherwise, the sphygmomanometer adopts an air inflation cuff worn on a user's arm to carry out the measurement.

The aforementioned prior arts all use a sensor to detect physiological signals, and then transmit them to a device to carry out a record analysis. However, a user must carry the device with him, causes his actions to be negatively affected. In addition, the aforementioned device is provided with a physiological information recording function, but it is very inconvenient for data backup or data transfer to another platform.

To a white-collar worker today, everyday he or she operates a computer for a long time, and he or she suffers from mental and physical pressure. His or her blood pressure, electrocardiogram, or body temperature might be abnormal under long-time working and working pressure. However, the aforementioned physiological signal monitoring devices are difficult for him/her to use simultaneously while he/she is operating a computer, or may even interfere with the operation of the computer. Hence, the physiological status of the computer user cannot be monitored simultaneously while he bears mental and physical pressure such that adverse mental and physical influence will be caused due to working pressure.

### SUMMARY OF THE INVENTION

In view of aforementioned problems, the object of the present invention is to provide a method and a system for monitoring physiological status, for monitoring physiological status of a user of a laptop promptly when the user operates the laptop.

In order to achieve the object, the present invention provides a method for monitoring physiological status, which is applied in a laptop. A sensor is disposed on the laptop adjacent to a keyboard of the laptop. When a user operates the laptop, at least one wrist of the user can touch the sensor. According to the method, the sensor is started and reset at first. Then, a threshold value range is given to set an allowable physiological status. Next, the sensor is used to detect continuously whether a physiological signal is input. When a physiological signal is input, the physiological signal is obtained by the sensor and then converted into a numerical value by a detection circuit. The numerical value is compared with the threshold value range to obtain a comparison result and the numerical value is output with the comparison result. If the numerical value falls outside the threshold value range, an alert is issued to the user or a pre-determined remote person.

The present invention also provides a system for monitoring physiological status in accordance with the method mentioned above, for monitoring a physiological signal of a user. The system for monitoring physiological status includes a laptop, a sensor, and a detection circuit. The laptop executes detection software for comparing a numerical value of the physiological signal with a threshold value range, and outputting the numerical value and a comparison result. If the numerical value falls outside the threshold value range, the laptop issues an alert. The sensor is disposed on the laptop adjacent to a keyboard of the laptop, for contacting a wrist of the use to receive the physiological signal of the user when the user is operating the laptop. The detection circuit is disposed in the laptop and electrically connected to the sensor and the laptop. The detection circuit provided for converting the physiological signal to the numerical value.

Through the sensor disposed on the laptop adjacent to the keyboard, the user touches the sensor and operates the laptop at the same time, the user's physiological signal is detected simultaneously. The laptop executes detection software, and then can record, analyze and compare the numerical value converted from the physiological signal. That is, the physiological status of the user is monitored at the same time that he is operating the laptop, wearing of an extra monitor device that might influence the user's action is not required anymore.

### BRIEF DESCRIPTION OF THE DRAWINGS

The present invention will become better understood from the detailed description given below for illustration only, and thus is not limitative of the present invention, in which:

FIG. 1 is a perspective view of a system for monitoring physiological status according to an embodiment of the present invention;

FIG. 2 is a block diagram of the system for monitoring physiological status according to the embodiment of the present invention; and

FIG. 3 is a flow chart of a method for monitoring physiological status of the present invention.

### DETAILED DESCRIPTION OF THE PREFERRED EMBODIMENTS

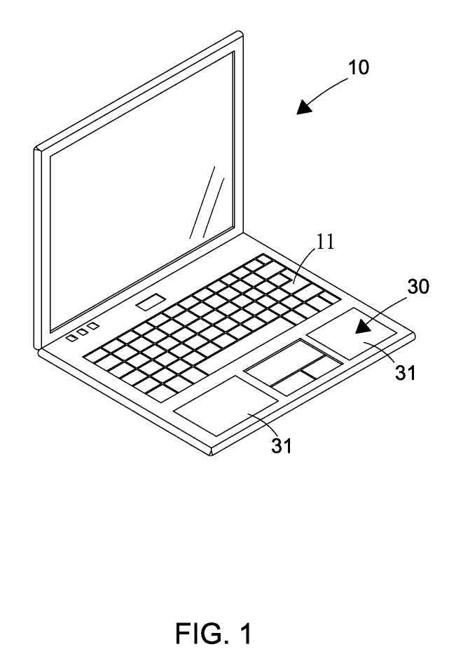

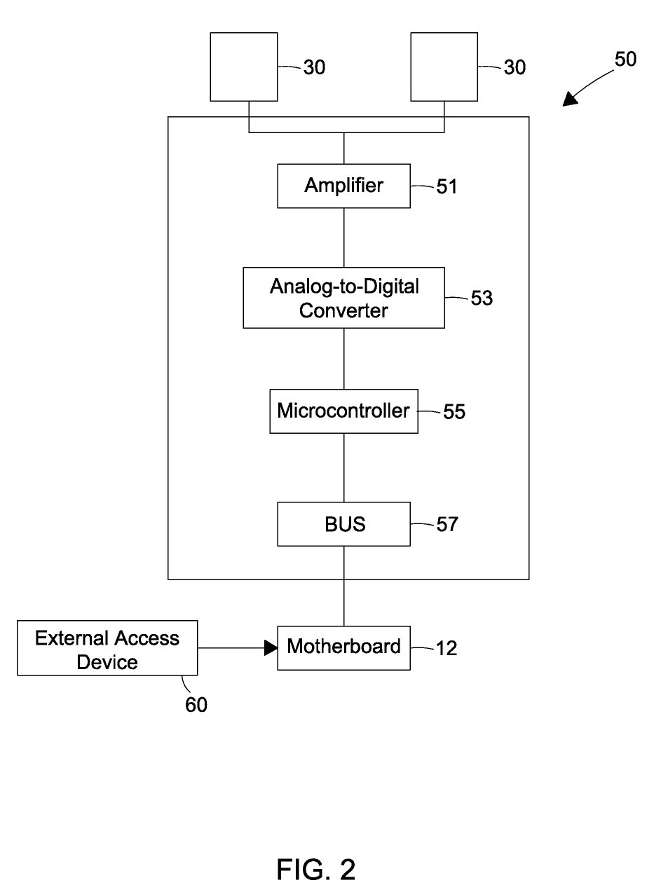

Referring to FIGS. 1 and 2, a system for physiological status according to an embodiment of the present invention is provided for monitoring a physiological signal of a user of a laptop, so as to determine whether an alert should be issued to remind the user to stop operating the computer temporarily.

The system includes a laptop 10, a sensor 30, a detection circuit 50, in which the sensor 30 and the detection circuit 50 are coupled to the laptop 10 for obtaining a numerical value of physiological signal of the user, and the detection circuit 50 transmits a numerical value to the laptop 10. The laptop 10 executes detection software, and a threshold value range is given and inputted to the detection software by the user. The laptop 10 receives the numerical value continuously and then outputs the numerical value. An output manner can be producing speech, displaying an image by the laptop 10, sending the numerical value to a remote terminal such as a medical center through a network, or storing the numerical value locally in portable storage media. At the same time, the laptop 10 compares the numerical value continuously with the threshold value range. The aforementioned detected physiological signal may be heartbeat rate, blood pressure, or electro-cardiogram variation rate, and is converted into the numerical value. The threshold value range lies in between an upper limit value and a lower limit value, and the upper limit value and the lower limit value can determined from a physiological information data base of the detection program according to the user's age and sexuality. The upper limit value and the lower limit value can also be input by the user himself.

The detection software mentioned above may compare a numerical value of heartbeat, blood pressure, body temperature, or electrocardiogram variation rate with the threshold value range. If the numerical value falls outside the threshold value range, it indicates that a user's physiological status might be abnormal. The laptop 10 can issue an alert, wherein the alert can be a caution sound, a caution image, or sending the comparison result and a caution message to a remote terminal through network. The remote terminal can be a medical center or a pre-determined person. The network can be the Internet, a general packet radio service (GPRS) network, global system for mobile communications (GSM) network, public switched telephone network (PSTN), 3rd generation (3G) network, or 3.5th generation (3.5G) network.

The sensor 30 is disposed on the laptop 10 adjacent to a keyboard 11 of the laptop, wherein the sensor 30 is provided for contacting a user's wrist when the user operates the laptop 10. The number of the sensors 30 can be two, for contacting both the user's left and right hands respectively at the same time. The sensor 30 detects the user's hypodermic vibration or weak current to obtain a required physiological signal, wherein, the sensor 30 is an electrode, temperature sensor or blood pressure transducer to detect the user's physiological signal. That is, the type of the sensor 30 is determined according to the type of the physiological signal required to be detected.

The aforementioned electrode is used to detect weak hypodermic currents, so as to obtain heartbeat, or an electro-cardiogram variation rate. The electrode is mainly used for providing a large contact area with the user's skin, wherein the electrode is a metal sheet or conductive rubber sheet 31. A conductive rubber sheet 31 is used as an example in the embodiment, but the selection of the electrode is not limited to conductive rubber sheet.

The laptop 10 stores the obtained numerical value in a hard disk, and when a next numerical value is obtained, a physiological status curve can be derived according to two or more numerical values, wherein the physiological status curve is used to determine whether the user's physiological condition is abnormal or not. The obtained numerical value(s) can be accessed by an external access device 60 such as a thumb drive, card reader, optical disk, or physiological signal detection device having a connection interface, thus the numerical value(s) can provide information for later medical analysis. After the medical analysis, the doctor can recommend a medical treatment or suggestion.

Referring to FIGS. 1 and 2 again, the detection circuit 50 is disposed in the laptop 10, and connected to the sensor 30 and a motherboard 12 of the laptop 10. The detection circuit 50 is used for converting the physiological signal obtained by the sensor 30 into the numerical value. The detection circuit 50 includes an amplifier 51, an analog-to-digital converter 53, a microcontroller 55, and a bus 57. The amplifier 51 is connected to the sensor 50, for amplifying the physiological signal obtained by the sensor and filtering noise. The analog-to-digital converter 53 is connected to the amplifier 51 for converting the physiological signal to a numerical value. The microcontroller 55 is connected to the analog-to-digital converter 53, for receiving the numerical value and transmitting the numerical value to the motherboard 12 of the laptop 10 through the bus 57 with a protocol, then the detection software executing laptop 10 compares the numerical value with the threshold value range.

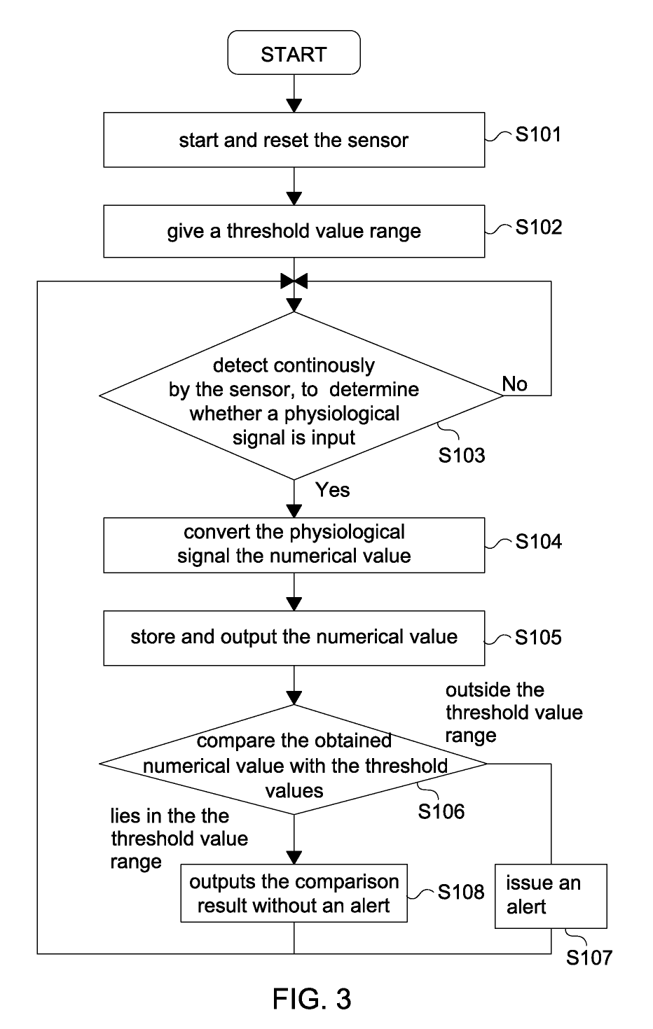

Referring to FIG. 3, a method for monitoring physiological status according to the present invention is applied in a laptop, wherein a sensor 30 is disposed on the laptop 10 adjacent to a keyboard 11 of the laptop 10. According to the method, start the detection software of the laptop 10 at first, so as to start and reset the sensor 30. The sensor 30 is set return-to-zero, so as to allow the sensor 30 to start detecting a physiological signal (S101).

After starting the detection software, a user profile is loaded, or a user inputted personal information, so as to give a threshold value range between an upper limit value and a lower limit value (S102), wherein the threshold value range is provided to be compared with the numerical value of the physiological signal (S102). The upper limit value and the lower limit value is provided by physiological data built in the detection software depending on the user's age and sexuality, and may also be input by the user himself.

Next, detect continuously by the sensor 30, to determine whether a physiological signal is input (S103). If the physiological sensor 30 detects no physiological signal, the next action is not carried out if the user does not touch the sensor 30. If the user touches the sensor 30 and the physiological sensor 30 detects an input physiological signal, the next action is carried out. The aforementioned physiological signal may be blood pressure, heartbeat or body temperature, but is not limit to these.

After the user touches the sensor 30, the physiological signal detecting device detects the input physiological signal, the sensor 30 obtains the physiological signal and the detection circuit 50 converts it to a numerical value (S104). Thereafter, the numerical value is transmitted to the laptop 10 to allow the laptop 10 to store the numerical value (S105), and the detection software carries out a comparison. The numerical value can be output as soon as the numerical value of the physiological signal is obtained, wherein the numerical value output method is producing speech by the laptop 10, or displaying an image displayed by the laptop 10 to remind the user the current physiological condition. Furthermore, the numerical value output method can be sending the numerical value to a remote terminal through a network to allow a remote medical system to record the numerical value.

The laptop 10 executing the detection software compares the obtained numerical value with the threshold values to obtain a comparison result (S106). The comparison result may be that the numerical value is larger than the upper limit value of the threshold value, the numerical value is smaller than the lower limit value of the threshold value, or the numerical value lies in between the upper limit value and the lower limit value.

The laptop 10 determines the comparison result. If the numerical value of the physiological signal falls outside the threshold value range, that is, the numerical value is larger than the upper limit value or smaller than the lower limit value, the laptop 10 issues an alert (S107). The alert is a caution sound produced by the laptop 10, or a caution image displayed by the laptop 10 to warn the user of the current abnormal physiological condition. The alert can also be sending the comparison result and a caution message to a remote terminal through the network, to seek assistance to prevent the user from being unable to call for help or lose his life because his physiological condition is not well and causes him to faint. If the numerical value of the physiological signal lies in the threshold value range, the laptop 10 outputs the comparison result without an alert (S108). This manner for outputting the comparison result is the same as the manner for issuing the alert.

According to the present invention, the physiological signal of the user of the laptop 10 is detected by the sensor 30 on the laptop 10 adjacent the keyboard 11. The laptop 10 executes detection software to compare a numerical value of a physiological signal with the threshold value range, and outputs the comparison result. The laptop 10 issues an alert if the numerical value falls outside the threshold value range. Obtaining the physiological signal of the user is simplified; no extra instruments that cause inconvenience to the user are required. By the present invention, additional time is not required for the user to obtain his/her physiological status and to prevent from disease.

It will be apparent to those skilled in the art that various modifications and variations can be made to the structure of the present invention without departing from the scope or spirit of the invention. In view of the foregoing, it is intended that the present invention covers modifications and variations of this invention provided they fall within the scope of the following claims and their equivalents.

What is claimed is:

1. A method for monitoring physiological status, applied in a laptop, wherein a sensor is disposed on the laptop adjacent to a keyboard of the laptop, the method comprising the following steps:
starting and resetting the sensor;
giving a threshold value range;
detecting continuously by the sensor, to determine whether a physiological signal is input;
obtaining the physiological signal by the sensor and converting the physiological signal into a numerical value;
comparing the numerical value with the threshold value range to obtain a comparison result; and
outputting the comparison result.

2. The method for monitoring physiological status as claimed in claim 1, further comprising the following step after the step of detecting whether a physiological signal is input:
storing the numerical value after obtaining the numerical value.

3. The method for monitoring physiological status as claimed in claim 1, wherein the threshold value range lies in between an upper limit value and a lower limit value.

4. The method for monitoring physiological status as claimed in claim 3, further comprising the following step before the step of outputting the comparison result:
determining the comparison result, and issuing an alert if the numerical values of the physiological signal falls outside the threshold value range.

5. The method for monitoring physiological status as claimed in claim 4, wherein the alert is caution sound produced by the laptop.

6. The method for monitoring physiological status as claimed in claim 4, wherein the alert is a caution image displayed by the laptop.

7. The method for monitoring physiological status as claimed in claim 4, wherein the alert is sending the comparison result and a caution message to a remote terminal through a network.

8. The method for monitoring physiological status as claimed in claim 1, further comprising the following step after the step of obtaining a numerical value of the physiological signal:
outputting the numerical value.

9. The method for monitoring physiological status as claimed in claim 8, wherein the step of outputting the numerical value is producing speech using the laptop.

10. The method for monitoring physiological status as claimed in claim 8, wherein the step of outputting the numerical value is displaying an image display by the laptop.

11. The method for monitoring physiological status as claimed in claim 8, wherein the step of outputting the numerical value is sending the numerical value to a remote terminal through a network.

12. A system for monitoring physiological status, for monitoring a physiological signal of a user, the system comprising:
a laptop executing detection software, for comparing a numerical value of the physiological signal with a threshold value range, outputting the numerical value and a comparison result, and issuing an alert if the numerical value falls outside the threshold value range;
a sensor disposed on the laptop, for receiving the physiological signal when the user is operating the laptop; and
a detection circuit, disposed in the laptop and connected to the sensor and the laptop, for converting the physiological signal to the numerical value.

13. The system for monitoring physiological status as claimed in claim 12, wherein the sensor is an electrode.

14. The system for monitoring physiological status as claimed in claim 13, wherein the electrode is a metal sheet.

15. The system for monitoring physiological status as claimed in claim 13, wherein the electrode is a conductive rubber sheet.

16. The system for monitoring physiological status as claimed in claim 12, wherein the sensor is a temperature sensor.

17. The system for monitoring physiological status as claimed in claim 12, wherein the sensor is a blood pressure transducer.

18. The system for monitoring physiological status as claimed in claim 12, wherein the detection circuit comprises:
an amplifier connected to the sensor, for amplifying the physiological signal;
an analog-to-digital converter connected to the amplifier, for converting the physiological signal into the numerical value; and
a microcontroller connected to the analog-to-digital converter, for receiving the numerical value and transmitting the numerical value to the laptop through a protocol range.
